(19) United States
(12) Patent Application Publication
(10) Pub. No.: US 2009/0270827 A1
(43) Pub. Date: Oct. 29, 2009

(54) Dressing

(76) Inventors: Boerge Gundersen, Tikoeb (DK); Brian Nielsen, Goerloese (DK); Klaus Budig, Oelstykke (DK); Chaabane Bougherara, Frederiksberg (DK)

Correspondence Address: JACOBSON HOLMAN PLLC, 400 SEVENTH STREET N.W., SUITE 600, WASHINGTON, DC, 20004, US

(21) Appl. No.: 11/992,187

(22) PCT Filed: Sep. 26, 2006
(86) PCT No.: PCT/DK2006/000524

§ 371 Date: Mar. 18, 2008

(30) Foreign Application Priority Data
Sep. 26, 2005 (DK) PA 2005 01340
Sep. 30, 2005 (DK) PA 2005 01369

(51) Int. Cl. A61L 15/22; B32B 5/20

(52) U.S. Cl. 604/369; 156/78

(57) ABSTRACT

A wound dressing comprising a backing layer and an absorbent layer, wherein the absorbent layer comprises a wound facing surface and a non-wound facing surface, said absorbent layer comprises discrete foam pads covering at least 50% of the wound facing surface of the absorbent layer and wherein the non-wound facing surface of the foam pads are attached to the backing layer. The structure of the dressing renders it possible for the foam to expand during wetting without changing the external dimensions of the absorbent layer substantially.

### BACKGROUND OF THE INVENTION

1. Field of the Invention

The invention relates to wound dressings, especially to dressings for highly exuding wounds.

2. Description of the Related Art

In the treatment of chronic wounds such as pressure sores and leg ulcers, absorbent wound dressings are used. Due to their high absorption capacity and pleasant skin-feel, dressings comprising absorbent foam may often be used. However, a major problem with foam dressings is the fact that many foams expand when wetted, and often the expansion in volume is up to hundred percent. Such expansion may give rise to problems. Pressure marks in the skin appear when the foam expands and the edge of the foam ends up folded into a double layer. This happens when the foam is not allowed to expand freely, and there is no space left for expansion. If the foam expansion does not result in a double layer, the build up stress in the foam may induce an undesired pressure against the wound site.

Stress from expanding foam in a dressing may affect the entire product design; cause the adhesive to detach from the skin, resulting in leakage. Furthermore, stress may induce undesired pressure to the skin or wound site.

Wound dressings comprising absorbent material such as foam, alginates or super absorbent material are well known in the art. Foam and other absorbent materials may be able to absorb up to 10 times their own weight, thus facilitating control of the wound exudates, even when handling highly exuding wounds. But when such absorbent materials, especially foam, absorb liquid, the volume of the material may expand. The foam may typically expand in the direction where expansion is allowed, which often results in an elevated pressure against the wound.

During the expansion stress may be built up in the foam and in the rest of the dressing. The stress may give rise to tensions and torsions and pressure in the foam, thus dragging the dressing in one or more directions. This may result in a less optimal fit of the dressing as well as an elevated risk of unintended detachment or displacement of the dressing.

Furthermore, application of a foam dressing to a protruding body part may be difficult, due to the thickness and stiffness of the dressing. The result may be voluminous and bulky and may give rise to pressure sores.

From EP patent application No. 575 090 is known a product suitable for absorbing wound exudates. The product comprises series of interconnected, perforated bags containing discrete beads of alginate. The beads may be threaded on a string. The product is especially suitable for use as a cavity filler, whilst it would be too lumpy for use as a flat dressing, as the beads are not fixed in the product and thus may move around in the bag, and enter into clusters giving rise to pressure marks.

International patent application No. WO 97/11658 discloses a wound dressing for irregularly shaped wounds, e.g. deep wounds. The wound dressing comprises a bag or pouch containing loosely packed absorbent material, such as foam pieces. The bag may be modeled to fit the wound cavity. The bag may also be used as a flat dressing, but as the pieces of foam may move freely in the bag, the risk of developing bulky portions, increasing the risk of pressure marks, is severe.

German patent application No. DE 100 54 928 A1 discloses an absorbent article comprising multiple layers of films, partly coated with absorbent particles. The film layer has an adhesive surface whereto a coat of particles is applied. The particles are coated in a pattern, e.g. like a chessboard, with areas without particles, serving as room for expansion when wetted. Each layer is sealed to another layer in order to encapsulate the absorbent particles and avoid free particles e.g. in a wound.

Attaching the absorbent material to a backing film by the use of adhesive may not be advantageous as the adhesive may loose its tack when wetted and thus the absorbent material will float freely and cause problems with double layers or agglomerates, thus causing pressure marks to the fragile skin of the patient.

WO 95/14451 disclose a wound dressing comprising an absorbent foam layer, the foam layer being provided with indentations for enhancing the flexibility of the dressing as well as provide folding- or cutting-lines of the dressing. The indentations are only extending partly through the foam layer and the raised portions of the foam are not able to move independently of each others.

GB 776,995 discloses a foam dressing wherein the foam layer has been provided with slits, facilitating stretching and bending of the foam. The slits leave little or no room for expansion of the foam when the dressing is in relaxed position.

EP 585 657 discloses a wound dressing comprising concentric foam rings, facilitating adaptation the dressing to the size of the wound. The foam rings lies next to each others, leaving no room for expansion of the foam during wetting.

Thus there is still a need for a wound dressing capable of absorbing large amounts of wound exudates without expanding in an uncontrolled manner.

### SUMMARY OF THE INVENTION

One object of the invention is to provide an absorbent dressing that substantially keeps its external dimensions while wetted.

Another object of the invention is to provide a flexible foam dressing which can be applied to curved body parts.

Yet another object of the present invention is to provide a dressing that does not become bulgy when wetted.

Still another object of the invention is to provide a dressing that reduces the risk of developing pressure marks.

### BRIEF DESCRIPTION OF THE DRAWINGS

The invention is disclosed more in detail with reference to the drawings in which

FIG. 1 shows an embodiment of the invention seen from below,

FIG. 2 shows the same embodiment in cross-section,

FIG. 3 shows the same embodiment of the invention in expanded state,

FIG. 4 shows another embodiment in expanded state,

FIGS. 5a-c show different patterns of the absorbent layer of the dressing,

FIG. 6 shows yet another embodiment of the invention,

FIG. 7 shows still another embodiment of the invention,

FIG. 8 shows yet another embodiment of the invention and

FIG. 9 shows an embodiment of the invention with bevelled edges.

### DETAILED DESCRIPTION OF THE PRESENT INVENTION

The invention relates to a wound dressing comprising a backing layer and an absorbent layer, wherein the absorbent layer comprises a wound facing surface and a non-wound facing surface, said absorbent layer comprises discrete foam pads covering at least 50% of the wound facing surface of the absorbent layer and the non-wound facing surface of the foam pads are attached to the backing layer wherein the wound facing surface of the absorbent layer comprises discrete foam pads surrounded by free space.

Double layer foam, i.e. folded foam developed during expansion of the foam, and hereby induced pressure marks, are prevented by designing the foam pad as a plurality of individual foam pads arranged in a pattern.

The discrete foam pads are surrounded by free space, thus leaving a spare volume for the foam to expand into. A high flexibility of the dressing is also achieved. The discrete foam pads are arranged with a distance to each other's.

In one aspect of the invention the distance between the discrete foam pads may be at least the same as the thickness of the foam pads.

The absorbent layer may further comprise material that does not hinder the expansion of the foam pads.

The distance between the individual pads allows expansion of each little pad, avoiding stress transmission to the remaining product design.

The backing layer may be any suitable material for serving as a carrier of the absorbent layer. In one embodiment of the invention the backing layer may be a water impervious but vapor permeable film, such as e.g. a polyurethane, polyethylene, polyester or polyamide film or laminates thereof.

In another embodiment of the invention the backing layer may be a permeable layer such as a net, gauze or non-woven. Such dressing may be combined with a secondary dressing such as an adhesive cover layer. The main function of the backing layer in this case is to control the position of the foam pads and serve as a carrier layer.

The backing layer may be provided with an adhesive layer on the wound-facing surface. The adhesive layer may cover the entire wound-facing surface of the backing layer or it may only cover at part of the surface. In one embodiment of the invention the adhesive layer constitutes an adhesive flange surrounding the absorbent layer.

The adhesive layer may be continuous or discontinuous, e.g. perforated or coated in a pattern.

The backing layer may be substantially the same size as the absorbent layer or it may be larger, thus providing the dressing with a flange of backing layer surrounding the absorbent layer like an island dressing. Said flange may be provided with a skin-friendly adhesive layer for attaching the dressing to the skin.

The foam may be any suitable expanding foam for wound care devices. It is preferred that the foam pads are made of a polymer based foam. In a preferred embodiment of the invention the foam is polyurethane foam.

In one embodiment of the invention the absorbent layer may comprise different kinds of foam. Due to the structure with multiple foam pads it is easy to mix different foam types in one dressing to obtain the desired properties.

The foam pads may be of any shape and configuration suitable for attachment to the backing layer. Preferably the foam pads are prepared from a foam sheet of substantially uniform thickness. The thickness of the foam pads may be 0,5-8 mm, more preferred 1-6 mm even more preferred 2-5 mm and most preferred 3-4 mm.

The foam pads may be of equal sizes or they may have individual size, e.g. smaller pads at the center of the dressing and larger pads at the edge portion. In one embodiment of the invention the edge portion constitutes a single pad encircling the central portion. The encircling pad facilitates a leak-proof barrier by absorbing exudates.

The absorbent layer may be prepared from a combination of foam pads of different thickness and/or sizes.

In one embodiment of the invention the discrete foam pads are having an edge portion and a central portion, wherein the density of the foam material is higher at the edge portion of the foam pad than it is at the central portion of the foam pad.

The edge portion of the foam pads may be substantially perpendicular to the wound-facing surface or the edge portion may be bevelled to the contour of a sloping line or curve.

The edge portion of a bevelled foam pad is defined as the portion of the pad where the foam has a reduced thickness compared to the central portion of the foam.

The edge portion of a non-bevelled foam pad is defined as the outmost 1 cm of the rim of the foam, such as the outmost 0.8 cm, the outmost 0,7 cm, the outmost 0,6 cm, the outmost 0,5 cm, the outmost 0,4 cm, or even the outmost 0,3 cm of the rim of the foam.

In one embodiment the absorbent layer has a density of between 100 and 400 kg/m³, such as between 120 and 300 kg/m³, or between 130 and 250 kg/m³ or even between 140 and 225 kg/m³. In a particular preferred embodiment of the invention the density is between 150 and 200 kg/m³.

The density of the foam material at the edge portion may in one embodiment of the invention be 50% higher than the density of the foam material at the central portion of the foam pads.

Measurement of density is common practice. Here, density should be measured under conditions of typical use that is at a temperature of 20° C., air pressure of 1013 hPa and relative humidity of 40%. Under those conditions a sample of the foam material is measured to determine the volume V and weighted to determine the mass m, and the density d calculated as d=m/V. It is central to the present invention that the edge portion of the dressing has a higher density than the central portion of the dressing. As foam with increased density will tend to cause gel blocking of the edge portion of the dressing, the tendency to leak is markedly reduced. In one embodiment of the invention the density of the foam material at

the edge portion is 50% higher than the density of the foam material at the central portion of the foam dressing, such as 75% higher, or double density, even up to five times higher.

The central portion of the foam pads may have a density of between 100 and 400 kg/m³.

Strings of absorbent material may interconnect the discrete foam pads. The strings may facilitate handling of the absorbent layer during production by controlling the position of the foam pads as well as the strings may act as a liquid barrier or guide. The strings may define closed compartments between the discrete foam pads, thus preventing wound exudates from spreading.

The strings are in the form of tiny pieces of absorbent material and has a negligible volume, thus they do not disturb the expansion of the foam. The strings may have the same thickness as the foam pads or they may be thinner.

The shape of the pads may be any suitable, random or systematically. In a preferred embodiment of the invention all of the pads have the same size and configuration, such as squares, circles, puzzle pieces or other geometrical or non-geometrical figures, and the pads may be arranged in a pattern. In one embodiment of the invention the pads are in the form of squares and are arranged with equal distance to each other.

The presence of multiple foam pads makes it easier to design a dressing for different wounds, e.g. the thickness of the foam pads may be larger in the central portion of the dressing than in the edge portion in order to enhance absorption capacity.

Most of the known adhesives used for wound dressings suffer from the drawback that they may loose their adhesive tack when wetted. Loss of adhesive tack may result in detachment of the absorbent layer, and uncontrolled movements of this.

Furthermore, if the absorbent layer is in the form of multiple pads, these may rearrange and end up in double layers or clusters, with pressure marks as a consequence.

In the dressing of the present invention the foam pads may be attached to the backing layer by non-detachable means, i.e. permanent means, such as by lamination or welding. However, an adhesive which is unaffected by moisture may also be used for attachment of the foam pads.

In one embodiment of the invention a part of the non-wound facing surface of the foam pads may be attached to the backing layer. Preferably, the attachment is in the form of one or more welding seams, e.g. in the form of dots.

In an especially preferred embodiment of the invention each foam pad is attached in a single point, e.g. in the center of the pad. In this way the foam may stay in place and yet be able to expand freely in all directions, without being restricted by the attachment.

Foam dressings usually comprise a single foam sheet as absorbent layer. When the foam sheet absorbs wound exudates, it may expand, sometimes up to 50-100% in longitudinal direction which inevitably will result in buckles and double layers that may give rise to pressure sores. When applied to a curved body part the stress in the foam will be even higher, due to compression of the foam in the curved areas.

Having an absorbent layer where the foam is divided into multiple pads and arranged with a distance between each other's, a dressing with a high absorbance, high flexibility and low stress is achieved. The dressing will not be bulky when wetted, as the foam pads are able to expand more freely into the free spacing surrounding each pad. The foam pads are attached to the backing film, thus keeping them in position while they expand, thus avoiding risk of creating double layers and bulges.

By using multiple foam pads, attached to a backing layer, the expansion of the foam is controlled compared to prior art, where the foam pads are floating around in an uncontrolled manner or buckling and detaching from the adhesive. The foam pads expand into the free space between the pads, thus forming a continuous surface against the wound, and decreasing the risk of maceration of the surrounding skin. The now filled spacing between the pads may serve as slits, providing enhanced flexibility of the dressing as well as they reduce the stress of compression in the dressing.

The preferred distance between the foam pads depends among other things on the properties of the foam. Foam with high expansion may demand higher distance than foam with lower expansion.

Foam used for wound care may usually have an expansion of 25-50% v/v, dependent on the type of foam. In a preferred embodiment of the invention the foam has an expansion of 30-40% v/v when fully wetted.

The amount of exudates present in the wound may also influence on the design of the dressing, highly exuding wounds may rapidly saturate the foam, thus obtaining maximum expansion, while less exudates may cause less expansion.

In one embodiment of the invention the foam pads may be arranged with a substantially equal distance to each other's.

In another embodiment of the invention the distance between the foam pads at the central portion of the dressing may be different from the distance between the foam pads at the edge portion of the dressing.

The foam pads may be placed with a higher distance between each others in the central, wound covering area, where a high expansion is expected, while the edge portion is in the form of pads with a narrow distance or it may be In the form of a single pad, encircling a multi-pads central portion.

When the dressing of the present invention is wetted, the foam pads may expand, but the spacing between the foam pads absorbs the expansion of volume. The external dimensions of the absorbent layer will remain substantially the same. When the absorbent layer maintains its size during absorption, the backing layer and optionally an adhesive flange surrounding the absorbent layer are not subjected to stress and torsion from the absorbent layer. Such stress may lead to leakage and detachment of the adhesive from the skin and is highly undesired.

The absorbent layer may typically have a width and length being much larger than the thickness of the layer, rendering the expansion of the foam in vertical direction (thickness) negligible compared to the expansion in horizontal direction (width and length). The volume expansion of the absorbent layer may preferably be less than 25%, more preferred less than 20%, even more preferred less than 15% and most preferred less than 10%.

The volume expansion during wetting is determined by measuring the dimensions of the dry absorbent layer, then soaking the absorbent layer in 0.9% saline water at 25° C. until fully saturated (1 hour) and then measure the dimensions of the wet absorbent layer.

In order to ensure that the foam pads expands into a continuous surface, the foam pads may cover 50-90% of the surface of the absorbent layer, more preferred 50-80% and even more preferred 50-70% of the surface of the absorbent layer and most preferred 55-65% of the surface of the absorbent layer.

The area to be covered by the foam pads depends on the expansion of the chosen foam as well as the expected amount of exudates from the wound.

The dressing of the present invention may also be less expensive to produce, as less foam is used, as well as the smaller pads offers better utilization of the foam during production of the dressing.

Furthermore, when applied to curved body parts, the dressing of the invention has an increased flexibility and may follow the movements of the body better than standard dressings. The flexibility facilitates the application of the dressing, especially when a non-adhesive dressing is used, as the dressing, due to the foam free areas may naturally curve and follow the contours of the body part when applied. A traditional one-piece foam dressing will be substantially planar, independent of the surface it is applied to, and has to be forced into shape when a secondary bandage is applied, thus demanding an extra hand to hold the dressing during application of a secondary bandage.

When the dressing of the invention is bended around a body part the pads will approach each others, thus creating a substantially continuous surface against the wound. Due to the passages between the foam pads, the bended foam will not have the same stress or tension as if it was a single continuous pad of foam, and there may still be room for expansion of the foam.

The total surface of the foam in the dressing of the present invention offers a larger total surface than if it was a single foam sheet, and the larger surface promotes faster initial absorption as each foam pad may absorbs from five surfaces (wound facing surface and four side surfaces) instead of only one.

The dressing of the present invention may comprise one or more active ingredients, for facilitation faster wound healing. The active ingredients may be biologically or pharmaceutically active agent.

The pharmaceutical medicaments will either be incorporated in the wound dressing or migrate to the wound surface and promote its function.

Examples of such pharmaceutical medicaments includes a cytochine such as a growth hormone or a polypeptide growth factor such as TGF, FGF, PDGF, EGF, IGF-1, IGF-2, colony stimulating factor, transforming growth factor, nerve stimulating growth factor and the like giving rise to the incorporation of such active substances in a form being apt to local application in a wound In which the medicament may exercise its effect on the wound, other medicaments such as bacteriostatic or bactericidal compounds, e.g. iodine, iodopovidone complexes, chloramine, chlorohexidine, silver salts such as sulphadiazine, silver nitrate, silver acetate, silver lactate, silver sulphate, silver sodium thiosulphate, silver-zirkonium complex or silver chloride, zinc or salts thereof, metronidazol, sulpha drugs, and penicillin's, tissue-healing enhancing agents, e.g. RGD tripeptides and the like, proteins, amino acids such as taurine, vitamins such ascorbic acid, enzymes for cleansing of wounds, e.g. pepsin, trypsin and the like, proteinase inhibitors or metalloproteinase inhibitors such as Illostat or ethylene diamine tetraacetic acid, cytotoxic agents and proliferation inhibitors for use in for example surgical insertion of the product in cancer tissue and/or other therapeutic agents which optionally may be used for topical application, pain relieving agents such as Ibuprofene or other NSAIDS, lidocaine or chinchocaine, emollients, retinoids or agents having a cooling effect which is also considered an aspect of the invention.

The active ingredient may also comprise odour controlling or odour reducing material.

The structure of the dressing of the invention with multiple discrete foam pads renders it possible to provide only a part of the foam pads with an active ingredient, thus saving the, often expensive, active agent and avoiding the agent to be exposed to parts of the wound site where it is not desired.

The matrix structure of the absorbent layer opens up for the possibility of being able to design different characteristics of the individual pad; i.e. incorporate an active agent in the center pads and another active agent in the surrounding pads.

The mechanical characteristics of the pads may also be designed for the specific purpose; e.g. by having softer foam in the surrounding pads with less absorption and retention capacity.

The structure of the dressing of the invention opens up for a high flexibility and freedom in the construction of dressings as the foam pads may be combined from pads with different properties and dimensions.

The dressing may be prepared by cutting the discrete foam pads and arranging them on the backing layer, attaching them by adhesive or lamination with heat and/or pressure.

The invention also relates to a method of preparing a wound dressing comprising a backing layer and an absorbent layer, wherein the absorbent layer comprises a wound facing surface and a non-wound facing surface, wherein said absorbent layer comprises discrete foam pads covering from 50 to 90 percent of the wound facing surface of the absorbent layer, and the non-wound facing surface of the foam pads are attached to the backing layer and wherein the wound facing surface of the absorbent layer comprises discrete foam pads surrounded by free space, said method comprising the steps of:
a) providing a backing layer,
b) providing an uncured foam mixture on one surface of the backing layer
c) encapsulating the uncured foam between the backing layer and a supportive film
d) allowing the foam to rise
e) contouring the foam into multiple discrete foam pads before the foam and
f) curing the foam.

The contouring of the foam pads may be initiated during the rising of the foam, but it is preferred that the foam is allowed to rise to its final size before contouring.

The foam may also be casted directly onto the backing layer. In a typical scenario the uncured foam mixture is poured out on a PU-film in contact with a conveyer belt. The uncured foam mixture is encapsulated between the PU-film and a supportive film. The foam is allowed to rise and cure. However, before the foam mixture reaches the cured state, the foam is contoured into multiple discrete foam pads. The contouring of the material may be done in different manners, for example by kiss-cutting rolls. Kiss-cutting may be performed from one or both sides of the foam sheets, where the rolls have contoured shapes in order to introduce embossed shapes of the foam when the foam passes the kiss cutting rolls. If only one of the kiss-cutting rolls has a contoured shape, the contoured roll may be both the upper and the lower roll.

### DESCRIPTION OF THE PREFERRED EMBODIMENTS

The invention is now explained more in detail with reference to the drawings showing preferred embodiments of the invention.

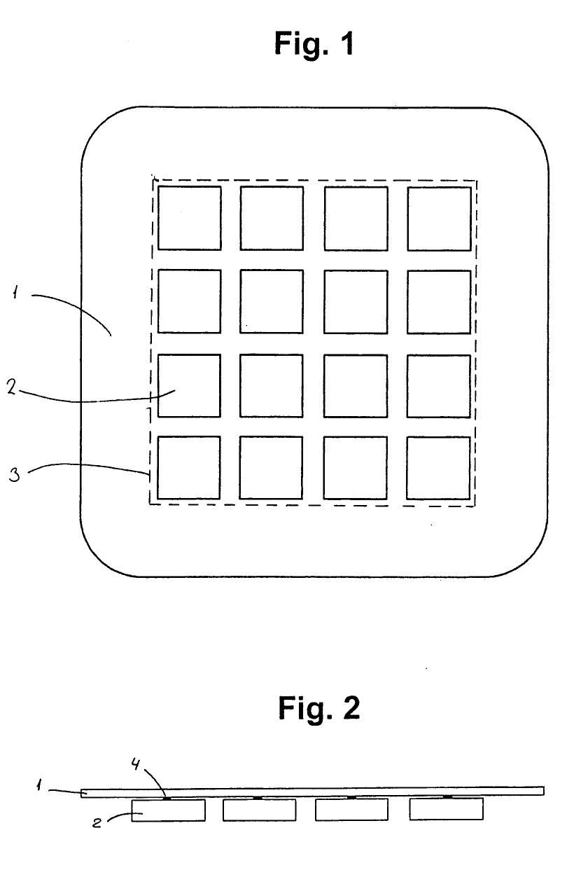

FIG. 1 shows an embodiment of the invention seen from below (wound-facing surface). The dressing comprises a backing layer (1) and an absorbent layer comprising a plurality of foam pads (2). The outline of the absorbent layer is indicated by the broken line (3). The foam pads (2) are arranged in a pattern, leaving a substantially equal distance between each pad (2).

FIG. 2 shows the same embodiment of the invention in cross-section, with the backing layer (1) and foam pads (2) attached thereto by non-detachable means (4) such as a welding. The attachment (4) is in the form of a single point at the center of each foam pad (2).

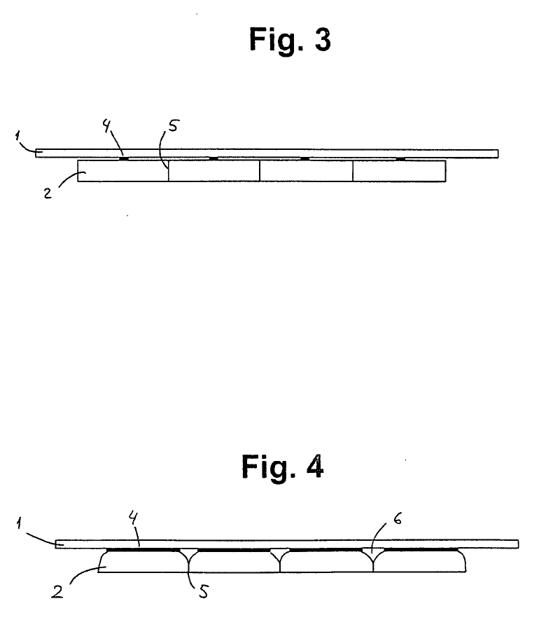

FIG. 3 shows the same embodiment after absorption of fluid and the resulting expansion. The foam pads (2) have expanded and now reach each others (5) to form a continuous surface against the wound.

In FIG. 4 is shown another embodiment of the invention wherein the entire non-skin facing surface of the foam pads (2) is attached to the backing layer (1) by non-detachable means (5). The Figure shows the dressing in expanded state. The foam pads (2) reach each other's at the skin-facing surface of the dressing to form a continuous surface. The presence of free space (6) near the attachment point may enhance the flexibility of the dressing, e.g. when bending the dressing.

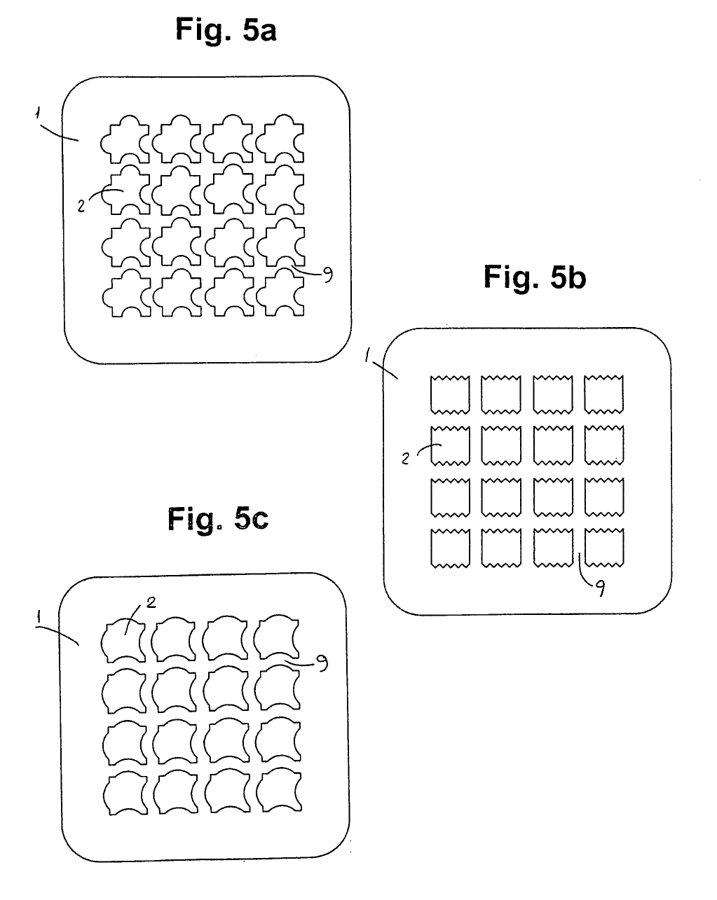

FIGS. 5a-5c show embodiments of the invention with different shapes of the foam pads (2). The shape of the pads as well as the pattern in which they are arranged may be any suitable for the purpose. The spacing (9) between the pads (2) may be in the form of rectilinear lines and may serve as a folding line as shown in FIG. 5b, which may sometimes be advantageous during application of the dressing. In other situations it is desired that no folding lines occur, like in FIG. 5a where the lines in the spacing (9) are broken by the pads (2).

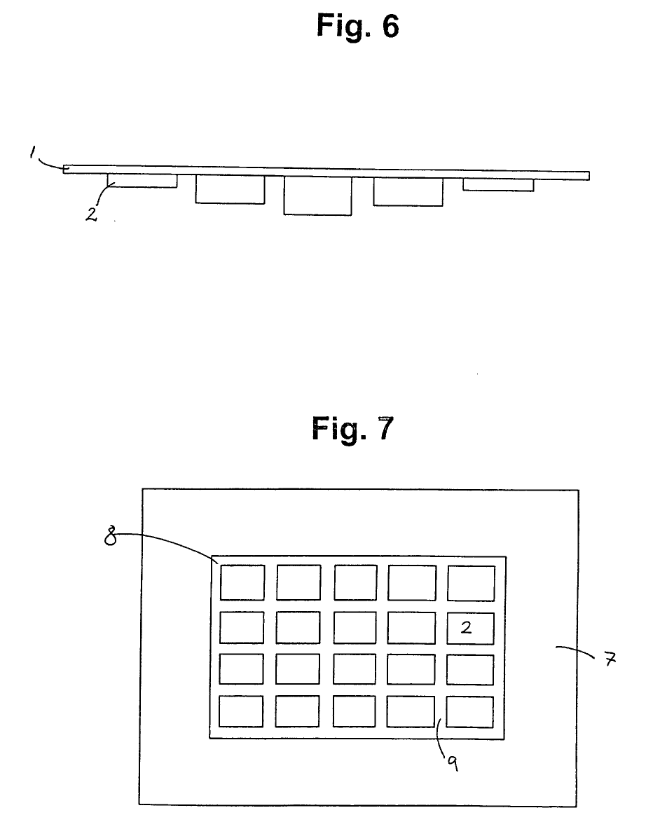

FIG. 6 shows an embodiment of the invention wherein the foam pads (2) has different thickness. The pads (2) are thicker at the center of the dressing, thus providing a better absorption right over the wound where the largest amount of exudates may be expected. Furthermore, the differentiated thickness may act as beveling of the absorbent layer, decreasing the risk of pressure sores being induced by a high edge of the foam.

FIG. 7 shows another embodiment of the invention wherein the absorbent layer comprises a central portion (8) with a plurality of foam pads (2) and an edge portion (7) comprising a single foam pad encircling the central portion (8). The central portion (8), which is exposed to exudates and thus high expansion, is in the form of multiple pads (2) arranged with a spacing (9) between in order to provide room for expansion, while the edge portion (7) may only be exposed to little or no exudates and thus little or no expansion may occur, and the edge portion (7) may primarily serve as a leakage barrier in order to avoid maceration.

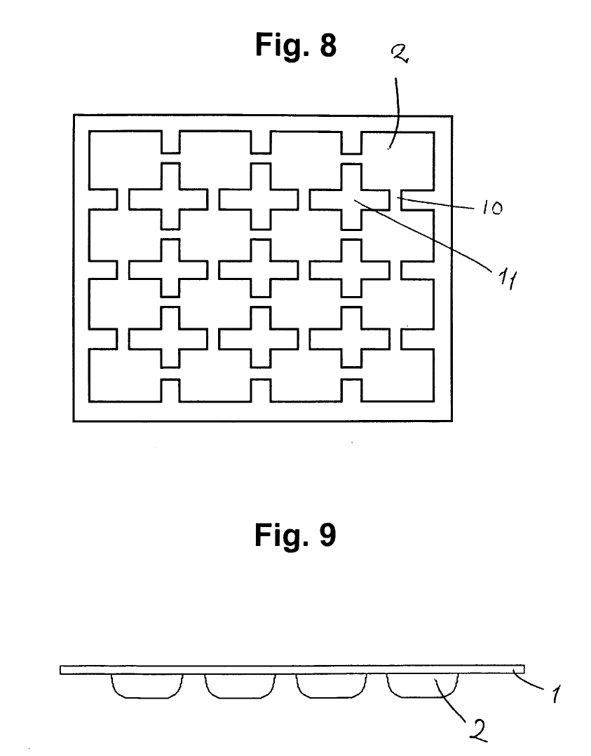

FIG. 8 shows an embodiment of the invention wherein the discrete foam pads (2) are interconnected by strings (10) of absorbent material. The strings facilitate control of the pads (2) during handling and production. Furthermore, the strings may define an enclosure (11) that may control the exudates and reduce the risk of leakage and maceration. The strings are preferably flexible and non-voluminous in order not to impair the movements of the foam pads during welting.

In FIG. 9 is shown another embodiment of the invention wherein the edge portions of the foam pads (2) are bevelled into a rounded contour.

1. A wound dressing comprising a backing layer and an absorbent layer, wherein the absorbent layer comprises a wound facing surface and a non-wound facing surface, wherein said absorbent layer comprises discrete foam pads covering from 50 to 90 percent of the wound facing surface of the absorbent layer, and the non-wound facing surface of the foam pads are attached to the backing layer and wherein the wound facing surface of the absorbent layer comprises discrete foam pads surrounded by free space.

2. A wound dressing according to claim 1 wherein the discrete foam pads are covering from 50 to 80 percent of the wound-facing surface of the absorbent layer.

3. A wound dressing according to claim 1 wherein the discrete foam pads are covering from 50 to 70 percent of the wound-facing surface of the absorbent layer.

4. A wound dressing according to claim 1 wherein the discrete foam pads are covering from 55 to 65 percent of the wound-facing surface of the absorbent layer.

5. A dressing according to claim 1 wherein the foam pads are made from a polymer based foam.

6. A dressing according to claim 1 wherein the foam pads are made of polyurethane.

7. A dressing according to claim 1 wherein the thickness of the foam pads is larger in the central portion of the dressing than in the edge portion.

8. A wound dressing according to claim 1, wherein the discrete foam pads are having an edge portion and a central portion, wherein the density of the foam material is higher at the edge portion of the foam pad than it is at the central portion of the foam pad.

9. A wound dressing according to claim 8, wherein the central portion of the foam pads has a density of between 100 and 400 kg/m³.

10. A wound dressing according to claim 8, wherein the density of the foam material at the edge portion is at least 50% higher than the density of the foam material at the central portion of the foam pads.

11. A wound dressing according to claim 8, wherein the edge portion is a bevelled edge portion.

12. A dressing according to claim 1 wherein the foam pads are attached to the backing film by welding.

13. A dressing according to claim 1, wherein the foam pads are attached to the backing film by lamination.

14. A dressing according to claim 1 wherein a part of the non-wound facing surface of the foam pads are attached to the backing layer.

15. A dressing according to claim 1 wherein all of the non-wound facing surface of the foam pads are attached to the backing layer.

16. A dressing according to claim 1 wherein the foam pads are arranged with a substantially equal distance to each other's.

17. A dressing according to claim 1 wherein the distance between the foam pads at the central portion of the dressing is different from the distance between the foam pads at the edge portion of the dressing.

18. A dressing according to claim 1 wherein the backing layer is a water impervious but vapor permeable film.

19. A dressing according to claim 1 wherein the backing layer is a net, gauze or non-woven.

20. A dressing according to claim 1 wherein the backing layer is provided with an adhesive layer on the wound-facing surface.

21. A wound dressing according to claim 1, wherein strings of absorbent material may interconnect the discrete foam pads.

22. A method of preparing a wound dressing comprising a backing layer and an absorbent layer, wherein the absorbent layer comprises a wound facing surface and a non-wound facing surface, wherein said absorbent layer comprises discrete foam pads covering from 50 to 90 percent of the wound facing surface of the absorbent layer, and the non-wound facing surface of the foam pads are attached to the backing layer and wherein the wound facing surface of the absorbent layer comprises discrete foam pads surrounded by free space, said method comprising the steps of:
a) providing a backing layer,
b) providing an uncured foam mixture on one surface of the backing layer
c) encapsulating the uncured foam between the backing layer and a supportive film
d) allowing the foam to rise
e) contouring the foam into multiple discrete foam pads before the foam and
f) curing the foam.
